(19) United States
(12) Patent Application Publication
(10) Pub. No.: US 2019/0168071 A1
(43) Pub. Date: Jun. 6, 2019

(54) Wearable Fitness Band For Measuring Body Parts

(71) Applicant: Visual Gains, LLC, Grapevine, TX (US)

(72) Inventor: Justin Paul Franks, Grapevine, TX (US)

(21) Appl. No.: 16/267,792

(22) Filed: Feb. 5, 2019

Related U.S. Application Data
(63) Continuation-in-part of application No. 16/138,828, filed on Sep. 21, 2018.
(60) Provisional application No. 62/561,538, filed on Sep. 21, 2017.

(51) Int. Cl. A63B 24/00; A63B 71/06; A61B 5/107

(52) U.S. Cl. CPC A63B 24/0062; A63B 71/0622; A61B 5/1072; A61B 5/1075; A63B 2024/0065; A61B 2503/10; A63B 2225/52; A63B 2230/60; A63B 2071/065; A63B 2071/0661; A63B 2220/836

(57) ABSTRACT

A wearable fitness band system for measuring a body part change in dimension utilizes a wearable fitness band device comprising strap assembly that is configured for being wrapped around the circumference of a body part to be measured. The strap assembly has a sensor that senses a change in dimension of the strap assembly while the strap assembly is wrapped around the body part and provides a sensor output. A processing unit coupled to the sensor receives the sensor output and provides a measurement output. A display that receives the measurement output and provides a measurement output reading correlating to the measurement output.

### CROSS-REFERENCE TO RELATED APPLICATIONS

This application is a continuation-in-part of U.S. patent application Ser. No. 16/138,828, filed Sep. 21, 2018, which claims the benefit of U.S. Provisional Application No. 62/561,538, filed Sep. 21, 2017, each of which is incorporated herein by reference in its entirety for all purposes.

### BACKGROUND

For bodybuilders, trainers, and other fitness-oriented individuals, one form of evaluating progression and goals is to track the physical changes in the size of a body part. The conventional approach typically includes using a fabric measuring tape similar to the ones used by tailors, to measure a body part and evaluate growth (or reduction as desired). Typically this is done pre-performance or post-performance of an exercise session. For the dedicated athlete however this may fall short of their needs. Some athletes would benefit from evaluating if an exercise/resistance level is providing enough muscle fiber engagement during performance to immediately react to the current level of exertion.

Currently there are no products currently available that provide such immediate and meaningful feedback regarding an individual's performance and the change in size of a particular body part. Improvements are therefore needed.

### BRIEF DESCRIPTION OF THE DRAWINGS

For a more complete understanding of the disclosure, reference is now made to the following descriptions taken in conjunction with the accompanying figures, in which:

FIG. 1 is front perspective view of a fitness band system during use by a user that includes a fitness band device for making body part measurements and a mobile device that is used with the fitness band system;

FIG. 2 a top plan view of the fitness band device of FIG. 1, shown with band spacers of different lengths for selectively shortening or lengthening the fitness band;

FIG. 3 is a block diagram of a representative data processing system of the fitness band system that may be used in illustrative embodiments of the invention;

FIG. 4 is a block diagram of the fitness band system in which illustrative embodiments may be implemented;

FIG. 5 is a bottom plan view of the fitness band device of FIG. 1, shown with a cutaway of a strap portion of the fitness band to reveal a band position sensor of the fitness band;

FIG. 6 is a front elevational view of the fitness band device of FIG. 1;

FIG. 7 is a plan view of a display of the mobile device of FIG. 1, shown with an exemplary screen of the mobile device display wherein recording measurements for a particular body part may be initiated;

FIG. 8 is a plan view of the display of the mobile device of FIG. 1 showing a screen of the display during recording of a measurement session;

FIG. 9 is a plan view of a chart history screen of the display of the mobile device of FIG. 1 during a chart-history mode of operation of the fitness band system;

FIG. 10 is a plan view of a history screen of the display of the mobile device of FIG. 1 during a session-history mode of operation of the fitness band system;

FIG. 11 is a plan view of a playback screen of the display of the mobile device of FIG. 1 during a session-playback mode of operation of the fitness band system;

FIG. 12 is a plan view of a goal-setting screen of the display of the mobile device of FIG. 1 during a goal-setting mode of operation of the fitness band system;

FIG. 13 is a plan view of a setting selection screen of the display of the mobile device of FIG. 1 during a setting-selection mode of operation of the fitness band system; and

FIG. 14 is a plan view of a band spacer selection screen of the display of the mobile device of FIG. 1 during a band-spacer-selection mode of operation of the fitness band system.

### DETAILED DESCRIPTION

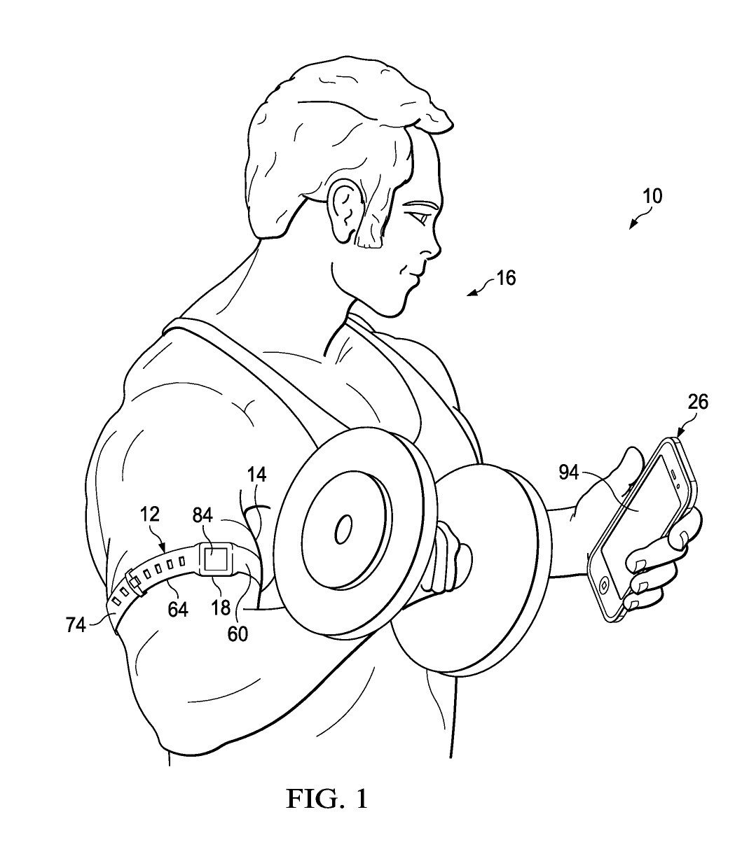

Referring to FIG. 1, a fitness band system 10 is shown with a wearable fitness band device 12 being worn on the bicep 14 of a user 16 while performing dumbbell curls as the user is holding a dumbbell. Although the fitness band 12 is shown being used on the user's bicep 16, it may be worn on other body parts of a user to measure changes in dimension of size of the particular body part on which it is worn. Furthermore, the fitness band device 12 may be worn during numerous different activities and is not limited to dumbbell curls, as is shown.

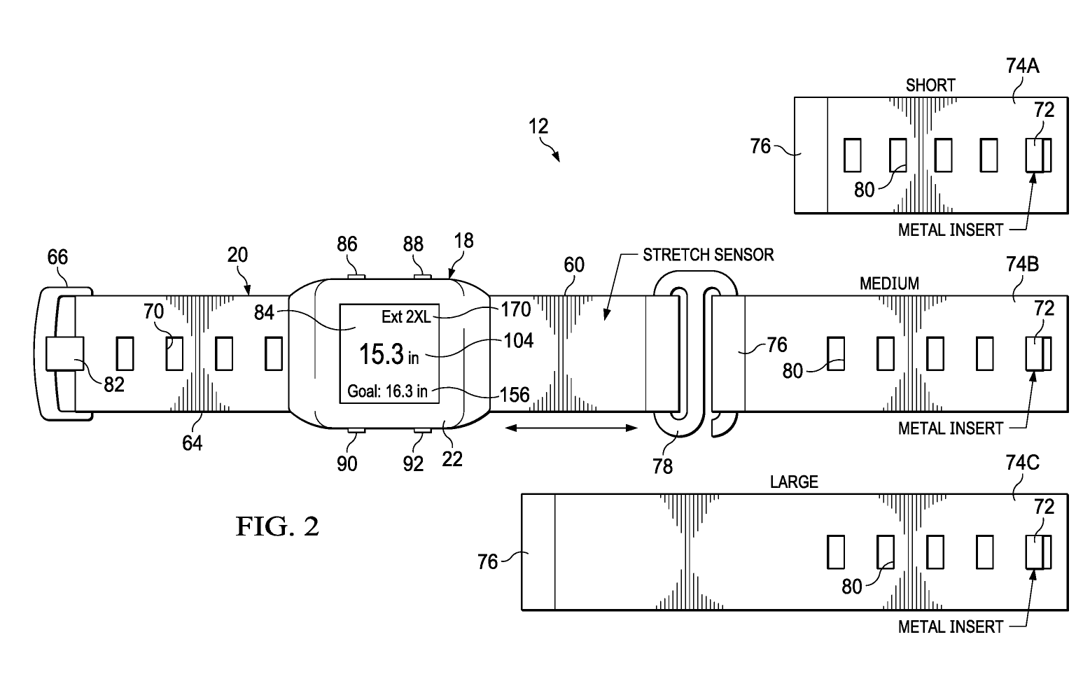

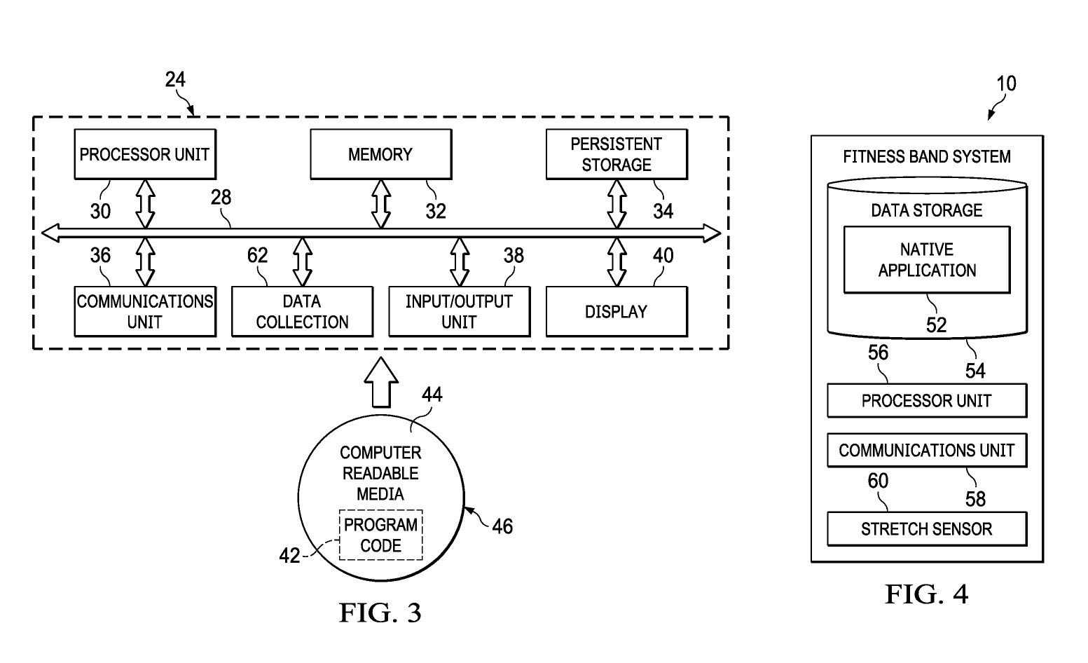

The fitness band 12 is configured to be wrapped around the circumference of a body part to be measured. As shown in FIG. 2, the band 12 includes an electronic device 18 secured to a strap assembly 20. The electronic device 18 includes a housing 22 for housing internal electronic components of the electronic device 18, such as a power source or battery, circuitry, processor, memory, etc. This may be in the form of a computer or data processing system 24, as shown schematically in FIG. 3.

The electronic device 18 may be self-contained and provided on the fitness band 12 itself, with all necessary components necessary to carry out the functions and tasks of measuring, providing feedback, recording, storing goals, etc., for full functioning and use of the fitness band 12, as it is described herein. In such instances, the fitness band 12 constitutes the entire fitness band system 10. In other instances, the electronic device 18 of the fitness band device 12 may be used in conjunction with a mobile device 26, as shown in FIG. 1. In such cases, the fitness band 12 and the mobile device 26 together may constitute the fitness band system 10. In other embodiments, the electronic device 18 of the fitness band device 12 is used with other separate devices, wherein the fitness band system 10 may include the fitness band device 12, the mobile device 26 and such other devices used in connection with the device 12.

The mobile device 26 can also include a data processing system, such as the data processing system 24 (FIG. 3) or a similar data processing system with all or less than all of the components described with respect to the processing system 24 of FIG. 3. The mobile device 26 may constitute a smartphone (e.g., iPhone® mobile phone, Android® mobile phone, etc.). In some embodiments, the mobile device 26 may be a laptop, a tablet computer, or any other portable electronic computing device having analogous components capable of providing similar functionality. The mobile device 26 may be in continuous or periodic communication with the electronic device 18, such as through a wireless (e.g., Bluetooth® wireless, Wi-Fi, cellular, etc.) or wired communication link for transmitting data between the mobile device 26 and the electronic device 18 of the fitness band device 12.

The block diagram of FIG. 3 shows the data processing system 24 in which illustrative embodiments may be implemented, either with the electronic device 18 of the fitness band 12 or the mobile device 26, or both. Other data processing or computer systems may also be employed with the fitness band 12 and/or mobile device 26. These may include a network of computers or computing devices, with the network being the medium used to provide communications links between various devices and computers connected together within the fitness band system 10.

The data processing system 24 is an example of a computer in which computer usable program code or instructions implementing processes described herein may be located. In this illustrative example, data processing system 24 provides a platform for the carrying out the various functions of the fitness band system and includes communications fabric 28, which provides communications between processor unit 30, memory 32, persistent storage 34, communications unit 36, input/output (I/O) unit 38, display 40, etc.

Processor unit 30 serves to execute instructions for software that may be loaded into memory 32. Processor unit 30 may be a set of one or more processors or may be a multi-processor core, depending on the particular implementation. Further, processor unit 30 may be implemented using one or more heterogeneous processor systems in which a main processor is present with secondary processors on a single chip. As another illustrative example, processor unit 30 may be a symmetric multi-processor system containing multiple processors of the same type.

Memory 32 and persistent storage 34 are examples of storage devices. A storage device is any piece of hardware that is capable of storing information, on a temporary basis and/or a permanent basis. Memory 32, in these examples, may be, for example, a random access memory or any other suitable volatile or non-volatile storage device. Persistent storage 34 may take various forms depending on the particular implementation. For example, persistent storage 34 may contain one or more components or devices. For example, persistent storage 34 may be a hard drive, a flash memory, a rewritable optical disk, a rewritable magnetic tape, or some combination of the above. The media used by persistent storage 34 also may be removable.

Communications unit 36, in these examples, provides for communications with other data processing systems, devices, and/or components. In these examples, communications unit 36 may be a network interface device. Communications unit 36 may provide communications through the use of either or both physical and wireless communications links (e.g., Bluetooth® wireless, Wi-Fi, cellular, etc.).

Input/output unit 38 allows for input and output of data with other devices or components that may be connected to data processing system 24. For example, input/output unit 38 may provide a connection for input through buttons, sensors, a keyboard, mouse, touchscreen, gesture commands, voice commands, etc. Display 40 provides a mechanism to display information to a user.

Instructions for the operating system and applications or programs may be located on persistent storage 34. These instructions may be loaded into memory 32 for execution by processor unit 30. The processes may be performed by processor unit 30 using computer implemented instructions, which may be located in a memory, such as memory 32. These instructions are referred to as program code, computer usable program code, or computer readable program code that may be read and executed by a processor in processor unit 30. The program code in the different embodiments may be embodied on different physical or tangible computer readable media, such as memory 32 or persistent storage 34.

Program code 42 is located in a functional form on computer readable media 44 that is selectively removable and may be loaded onto or transferred to data processing system 24 for execution by processor unit 30. Program code 42 and computer readable media 44 form computer program product 46 in these examples. In one example, computer readable media 44 may be in a tangible form, such as, for example, an optical or magnetic media that is inserted or placed into a drive or other device that is part of persistent storage 34 for transfer onto a storage device, such as a hard drive that is part of persistent storage 34. In a tangible form, computer readable media 44 also may take the form of a persistent storage, such as a hard drive, a thumb drive, or a flash memory that is connected to a data processing system. The tangible form of computer readable media 44 is also referred to as computer recordable storage media. In some instances, computer recordable media 44 may not be removable.

In many cases, program code 42 may be transferred to data processing system 24 from computer readable media 44 through a communications link to communications unit 36 and/or through a connection to input/output unit 38. The communications link and/or the connection may be physical or wireless. The computer readable media also may take the form of non-tangible media, such as communications links or wireless transmissions containing the program code.

In some illustrative embodiments, program code 42 may be downloaded over a network to persistent storage 34 from another device or data processing system for use within data processing system 24. For instance, program code stored in a computer readable storage medium in a server data processing system may be downloaded over a network from the server to data processing system 24. The data processing system providing program code 42 may be a server computer, a client computer, or some other device capable of storing and transmitting program code 42, which may located in a remote location.

The different components illustrated for data processing system 24 are not meant to provide architectural limitations to the manner in which different embodiments may be implemented. The different illustrative embodiments may be implemented in a data processing system including components in addition to or in place of those illustrated for data processing system 24. Other components shown in FIG. 3 can be varied from the illustrative examples shown.

FIG. 4 is a schematic block diagram of the fitness band system in which illustrative embodiments may be implemented. The fitness band system 10 may include the fitness band device 12 and/or cooperating mobile devices (e.g., mobile device 26) and/or other computer or data processing systems and their communication links. The electronic device 18 and/or mobile device 26, and/or any other computer or data processing system of the system 10 is provided with a client application in the form of a native mobile application 52. The native application 52 may be obtained from an application service provider (ASP) that provides the application software to the electronic device 18 of the fitness band 12 and/or mobile device 26. The application 52 may be part of a web-based system platform wherein functions described herein may be performed on other devices, through either a dedicated application or a web-browser interface. Users may provide login credentials to access and enter information and create user profiles on the fitness band system 10 that is then accessible from any device by providing the same login credentials.

The application 52 may be provided by the ASP either free of charge or for a fee. Furthermore, once the application 52 is purchased, use of the application 52 may be provided free of charge or the ASP may require the user to purchase a subscription or pay a licensing fee, which may be payable on a periodic basis, for the continued use of the application 52 or for access to the fitness band system 10.

As shown in FIG. 4, the native application 52 is stored in data storage 54 of the system 10. The data storage 54 may include memory (RAM) and persistent storage, with all or a portion of the native application 52 being stored on these. A computer processor unit 56 is provided with the system 10 for running programs and processing data. A communications module 58, including hardware and software, provides communications from and to the fitness band 12 through the use of either or both physical and wireless communications links, and allows the fitness band 12 to connect to a network.

The fitness band 12 of the system 10 is also provided with a sensor 60. The sensor 60 is that which senses a change in dimension of the wearable fitness band 12 or strap assembly 20 while the strap assembly 20 is wrapped around a selected body part. The sensor 60 allows the sensing and collecting of data related to the change in dimension of the strap assembly 20 that corresponds to the change in dimension to the body part on which it used. The native application 52 makes use of the sensor 60 to sense and collect sensor data that is then provided to one or more of the data collection modules 62 (FIG. 3) of the data processing system 24.

Referring to FIG. 2, the sensor 60 may be in the form of a stretch sensor that forms a part or section of the strap assembly 20 of the fitness band 12 and is coupled at one end to one side of the electronic device 18. The stretch sensor 60 is typically in the form of an elastic body that resiliently expands and contracts when force is applied to or removed from the elastic body. In most cases, the stretching force will be applied primarily from opposite ends of the elastic body, wherein its length is increased or decreased as force is applied or removed from the ends of the elastic body.

The stretch sensor 60 may be an elastic stretch sensor. Such sensors may be electronic sensors that provide a sensed data output relating to changes in electrical properties due to the stretching or deformation of the elastomeric sensor. Such elastic stretch sensors include those formed from flexible capacitors that provide sensed data output relating to a change in capacitance due to the change in the dimensions or deformation of the flexible capacitor. Such flexible capacitor sensors usually employ one or more elastomeric dielectric layers sandwiched between elastomeric electrodes. The change in capacitance may be due to changes in surface area of the elastomeric electrodes, thickness of the elastomeric dielectric, and/or electrode spacing, which result from the deformation of the elastomeric capacitor. Non-limiting examples of commercially available flexible capacitors are those stretch sensors available from StretchSense Limited, Auckland, New Zealand. In other embodiments, the stretch sensor may be in the form of a resistance sensing device, such as a strain gauge or other device where changes are measured by a change in the electrical resistance due to changes in shape or deformation due to applied force. Other stretch sensors capable of measuring changes in dimension and providing data or information output that relates to a change in dimension of a body part may also be used for the stretch sensor.

The components of the stretch sensor 60 may be encased in a stretchable or elastic fabric material, such as Spandex fabric, which may be colored, sized and configured to match or cooperate with the materials forming all or some portions of the strap assembly 20. The fabric material or other portions of the stretch sensor may be coupled to the housing 22 of the electronic device 18 though various coupling means. The total length of the stretch sensor 60 when it is in a fully retracted or non-stretched configuration may range from 0.5 inch to 8 inches, more typically from 1 inch to 4 inches. In particular embodiments, the total length of the stretch sensor 60 in a contracted or non-stretched configuration may be at least, equal to, and/or between any two of 0.5 inch, 1 inch, 1.5 inch, 2 inches, 2.5 inches, 3 inches, 3.5 inches, 4 inches, 4.5 inches, 5 inches, 5.5 inches, 6 inches, 6.5 inches, 7 inches, 7.5 inches, and 8 inches.

It should be noted in the description, if a numerical value, amount or range is presented, each numerical value should be read once as modified by the term “about” (unless already expressly so modified), and then read again as not so modified unless otherwise indicated in context. Also, in the description, it should be understood that an amount range listed or described as being useful, suitable, or the like, is intended that any and every value within the range, including the end points, is to be considered as having been stated. For example, “a range of from 1 to 10” is to be read as indicating each and every possible number along the continuum between about 1 and about 10. Thus, even if specific points within the range, or even no point within the range, are explicitly identified or referred to, it is to be understood that the inventor appreciates and understands that any and all points within the range are to be considered to have been specified, and that inventor possesses the entire range and all points within the range.

The stretch sensor 60 may be designed and configured to provide a linear change in dimension of up to 1.5 times or less of its contracted or non-stretched length. In certain instances, the stretch sensor 60 may be designed and configured to provide a linear change in dimension of at least, equal to, and/or between any two of 1.5, 1.4, 1.3, 1.2, 1.1, 1.0, 0.9, 0.8, 0.7, 0.6, or 0.5 times or less of its contracted or non-stretched length. In particular embodiments, the stretch sensor 60 may provide a change in dimension of from 4 inches or less. This will typically be a change in the length of the stretch sensor. In particular embodiments, the stretch sensor may provide a maximum change in length of from up to any one of 4 inches, 3.5 inches, 3 inches, 2.5 inches, 2 inches, 1.5 inches, 1 inch, 0.75 inch, or 0.5 inch. The stretch sensor 60 may provide a sensed data output in measured increments of 0.1 inch to 0.01 inch or less. The stretch sensor may measure a change in dimension of from 0.01 inch or 0.1 inch or more.

As shown in FIG. 2, one end of the stretch sensor 60 is physically coupled to the electronic device 18 of the fitness band. Electrical couplings are provided for receiving signals of sensed data output from the sensor 60 that are transmitted to and received by the electronic device 18, such as received by the data collection module 62 (FIG. 3) of data processing system 24. In other embodiments, the stretch sensor 60 may be positioned or spaced away from the electronic device 18 on the strap assembly 20, with transmission devices or electrical connections for transmitting the sensed data output from the sensor 60 to the data processing system 24 of the fitness band 12, the mobile device 26 or other computer or data processing system of the system 10.

The strap assembly 20 further includes a strap portion 64. The strap portion 64 is in the form of a relatively flat, flexible strap, although it may have other configurations, and is coupled at one end to the electronic device 18 or its housing 22 on the opposite side from the stretch sensor 60 through various coupling means. The other end of the strap portion 64 may be provided with a buckle assembly 66 or other fastener to facilitate rigid coupling to other components of the strap assembly 20 to secure the fitness band 12 around the user's body part being measured. The strap portion 64 should be non-stretchable. This may be achieved by forming the strap portion 64 from relatively non-elastic materials or materials having a low enough elasticity during normal use so that they do not readily stretch lengthwise significantly during use of the fitness band 12, which may otherwise interfere with the fitness band's accuracy.

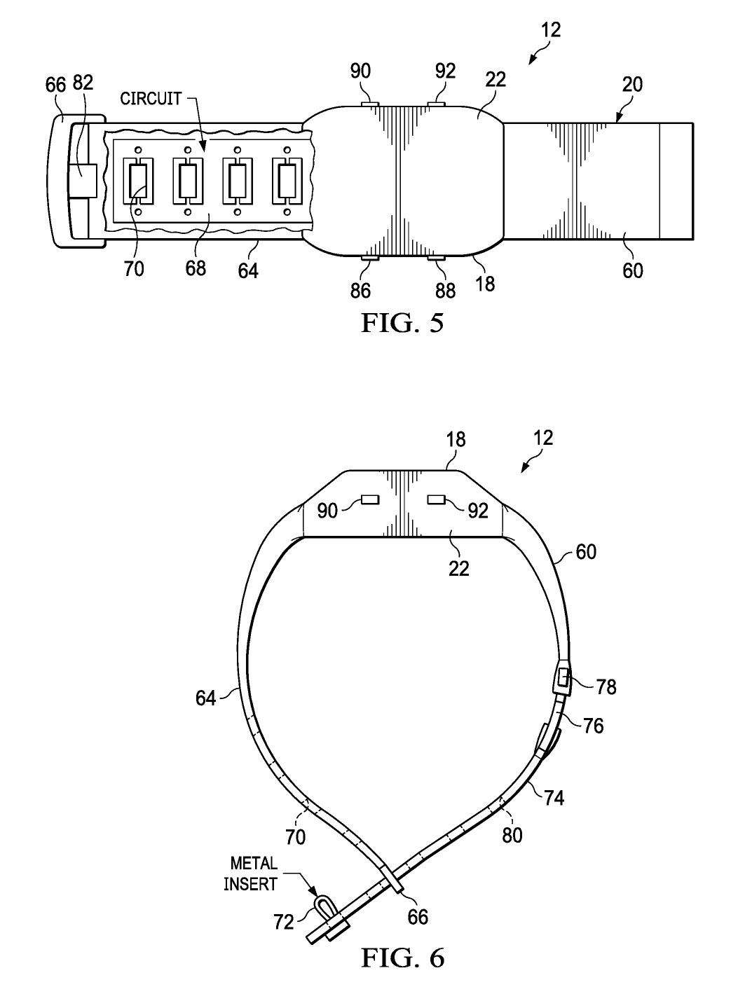

The strap portion 64 may be used to house or contain a band position sensor, such as the band position sensor assembly 68 shown in FIG. 5. As shown, the position sensor assembly 68 is sheathed or positioned between layers of the materials forming the strap portion 64. The band position sensor 68 is formed from a flexible circuit that is configured to bend and flex with the flexible strap portion 64. The circuit 68 may be configured as a simple potentiometer or other electrical device or sensor with linear or circumferentially spaced apart openings or apertures 70 or other structures for engaging or receiving a position coupling 72 (FIG. 6). Each of the openings or apertures 70 is spaced a known distance apart. Individual circuitry (not shown) provided on the circuit 68 connect each of these apertures 70 to the electronic device 18, which may provide electrical power to the circuit 68.

The position coupling 72 may constitute a circuit insert or contact element. The circuit insert or contact element 72 is a metal or electrically conductive element that is sized and configured to engage or be received within each of the openings or apertures 70. The apertures 70 are surrounded with an electrically conductive material (e.g., electrically conductive metal) but provide an open circuit in the circuitry associated with the aperture 70 when the aperture remains empty. When the insert 72 is inserted into an aperture 70, however, the circuit is completed to generate an electrical signal indicative of which aperture 70 the insert 72 is positioned that may be communicated to the data processing system 24. As is discussed later on, this allows a determination of the dimensions of the fitness band 12.

As shown in FIG. 2, removable band spacers 74A, 74B, and 74C may be provided with the fitness band 12. The band spacers 74 are each in the form of a relatively flat, flexible strap, although they may have other configurations. Like the strap portion 64, the band spacers 74A, 74B, 74C should be formed from non-stretchable material. This may include materials that are relatively non-elastic material or that have a low enough elasticity during normal use so that it does not stretch lengthwise significantly during use of the fitness band 12. By making all of the components of the strap assembly 20 other than the stretch sensor 60 from non-stretchable materials, only the stretch sensor 60 is allowed to stretch so that accuracy of the fitness band 12 is maintained.

Each band spacer 74A, 74B, 74C may be of a different known length, such as small, medium, and large, respectively, to selectively increase or decrease the length of the strap assembly 20. While in the embodiment shown there are three different band spacers 74, additional band spacers of different lengths or sizes may be provided with the fitness band 20 as well. Band spacers 74 of different sizes allow the fitness band 12 to accommodate body parts of different sizes. The band spacers 74 may differ in size by the amount of normal or maximum lengthwise deformation the stretch sensor 60 can undergo. For example, if the stretch sensor 60 can stretch or undergo a maximum change in length of up to 2 inches during normal use, each band spacer 74A, 74B, 74C may be 2 inches longer than the next size down to accommodate for the limits of stretch or deformation the stretch sensor can undergo.

This allows the band 12 to be used on different size body parts without overstretching the stretch sensor 60 or stretching it beyond its maximum limits. In particular embodiments, the fitness band 12, with or without any necessary band spacers 74, may be configured to be strapped or secured around a body part to be measured of from 8 inches to 50 inches, with from 10 inches to 25 inches being typical. In particular embodiments, the fitness band 12, with or without any necessary band spacers 74, may be configured to be strapped or secured around a body part to be measured of at least, equal to, and/or between any two of 8 inches, 9 inches, 10 inches, 11 inches, 12 inches, 13 inches, 14 inches, 15 inches, 16 inches, 17 inches, 18 inches, 19 inches, 20 inches, 21 inches, 22 inches, 23 inches, 24 inches, 25 inches, 26 inches, 27 inches, 28 inches, 29 inches, 30 inches, 31 inches, 32 inches, 33 inches, 34 inches, 35 inches, 36 inches, 37 inches, 38 inches, 39 inches, 40 inches, 41 inches, 42 inches, 43 inches, 44 inches, 45 inches, 46 inches, 47 inches, 48 inches, 49 inches, and 50 inches.

One end of each band spacer 74 is provided with a loop or other attachment portion 76 for removably or detachably coupling the end of the spacer 74 to the strap assembly 20. In the embodiment shown, a buckle or clip 78 is used to couple the band spacer 74 to the end of the stretch sensor 60 opposite the electronic device 18. Various other releasable fasteners may be used to releasably attach one end of the band spacer 74 to the stretch sensor 60 or other portions of the strap assembly 20.

The insert element 72 is coupled to the opposite end of the band spacer 74, as shown in FIGS. 2 and 6. The insert 72 is located at a known position on the band spacer 74. When the strap assembly 20 is positioned and worn on the body part to be measured, the insert element 72 of any one of the band spacers 74A, 74B, 74C, is inserted into one of the apertures 70 on strap portion 64. Because the spacing or distance of each of the apertures 70 of the position sensor 68 is known and the position of the insert 72 on the band spacer 74 is known, when the strap assembly 20 is secured to and worn on the body part of the user, the length of the strap assembly 20 can be known. Thus, changes in dimension of the stretch sensor 60 can be used to directly measure changes in the dimensions of the body part on which the device 12 is worn.

A series of holes or apertures 80 may also be provided in the band spacer for engaging the prong 82 of buckle 66 to facilitate securing of the strap assembly 20 on the body part being measured. In some embodiments, the buckle 66 may be eliminated and the position coupling or insert element 72 may be used to couple the band spacer 74 to the strap assembly 20.

In other embodiments, the removable band spacers 74 may be eliminated or a strap portion or band spacer 74 may be permanently or non-removably attached to the sensor 60 or other portions of the strap assembly 20. In one embodiment, the strap portion 64 is merely attached at one end to the electronic device 18 and at the other end to the stretch sensor 60, without the use of any band spacers and without requiring the circuit 68. In still other embodiments, the stretch sensor 60 is positioned between two strap portions that are secured at each end to the electronic device 18. In still other embodiments, all or a portion of the strap assembly 20 may be formed from the stretch sensor 60.

The various portions or components of the fitness band 12 (e.g., strap assembly 20, strap portion 64, band spacer 74, electronic device 18, electronic device housing 20, buckle 66, clip 78, etc.), other than the stretch sensor 60, may be non-stretchable so that they do not stretch lengthwise significantly during use of the fitness band 12, thus contributing to inaccurate measurements measure by the stretch sensor 60. The electronic device housing 20, stretch sensor 60, strap portion 64, band spacers 74, etc., or components thereof, may be formed from durable and sweat or water-resistant materials. Typically, these components or portions thereof may be formed of elastomeric or plastic material, such as nitrile butadiene, silicone, PVC, polyurethane, isoprene, neoprene, and the like. Other materials, such as fabric (e.g., nylon fabric), braided cordage, leather and other materials may be used provided they provide the non-elastic or limited stretching required.

As shown in FIG. 2, the electronic device 18 is provided with a display 84 for providing information to the user. The display 84 may be an LCD, OLED, or other type of display. In certain embodiments, instructions may be provided to the electronic device 18, such as by manual input by the user, to selectively provide a reverse, rotated, and/or upside-down or flipped image of the display 84. This may allow the user to flip or reorient the display image, such as after it is positioned on a particular body part, but where the information displayed is initially upside down or at a particular angle. Instead of reorienting the fitness band 12 so that the display image is right-side up or at the desired angle, the user may merely provide an instruction to reorient the image so that it is flipped or rotated (e.g., 180 degrees, 90 degrees, etc.) on the display 84 itself. In addition, instructions to the electronic device 18 may be provided to reverse the displayed image so that everything in the display 84 is shown as a mirror image. This may allow the user to view the display 84 as a reflection in a mirror so that information is properly oriented while viewing the mirror reflection of the display 84. The display 84 may also be selectively switched off in an incognito mode, so that no or only selected information is displayed on the display 84. This prevents others around from viewing information on the display 84 that the user may not want them to see. Accelerometers and/or gyroscopes (not shown) or other sensors may also be provided with the electronic device 18 to facilitate providing instructions, which may be automatic, for orienting the image of the display 84, as occurs with current smart phones. Such a feature may be disabled or enabled, as the user prefers.

One or more control buttons 86, 88, 90, 92 may be provided on the electronic device 18. These form at least a part of the input/output unit 38 (FIG. 3) and allow the user to control the functionality of the fitness band 12 and to input various instructions or commands to the data processor 24, such as adjusting the display 84, powering the device on and off, navigating to different screens, prompting and recording measurements, etc. In other embodiments, the display 84 may constitute a touch screen wherein touch input to the screen may be made to provide commands or instructions. Sensors or other devices may be provided with the electronic device 18 apart from any touch screen assembly so that input may be provided by tapping or touching the device 18 in a certain way at a particular location or providing a series or a pattern of such tapping or touching (e.g., tapping an edge of the housing 20, etc.) at such a location to provide instructions or commands. In still other embodiments, the electronic device 18 may include sensors, microphones, cameras or optical devices wherein non-touch input may be provided to the device 18 and processor 24. This may include gestures, movements, audio commands, etc., that are detected proximal to the device 18 without requiring touching the device 12.

As discussed previously, the electronic device 18 may communicate, such as through a wireless (e.g., Bluetooth® wireless, Wi-Fi, cellular, etc.) or wired communication link, with a separate mobile device, such as the mobile device 26, or another device, such as laptop or tablet computer, desktop computer, remote server, etc. In many instances, the fitness band 12 will be used in conjunction with a mobile device 26, such as a smart phone. For purposes of discussion, the use of the fitness band 12 with a smart phone mobile device will therefore be described. It should be readily apparent, however, that the fitness band 12 may be used by itself or with other devices, such as non-smart phone devices of laptops, tablets, desktop computers, etc., with similar functionality as that described with respect to the smart phone mobile device 26.

The mobile device 26 or other device may be provided with a native application, such as the native application 52 (FIG. 4) that provides functionality with and communication with the electronic device 18 of the fitness band 12.

The mobile device 26 is also provided with a display 94 for displaying information. The display 94 may constitute a touch screen so that instructions and commands can be input to the mobile device 26. Other control buttons, sensors, microphones, cameras, optical devices, etc. may be provided with the mobile device 26, for inputting commands and instructions to the mobile device 26. In an alternative embodiment, the electronic device 18 of the fitness band 12 may not be provided with the display 84. Data or information from or collected by the fitness band 12 may be communicated to the mobile device 26 and such information may be displayed on the display 94 of the mobile device 26 instead.

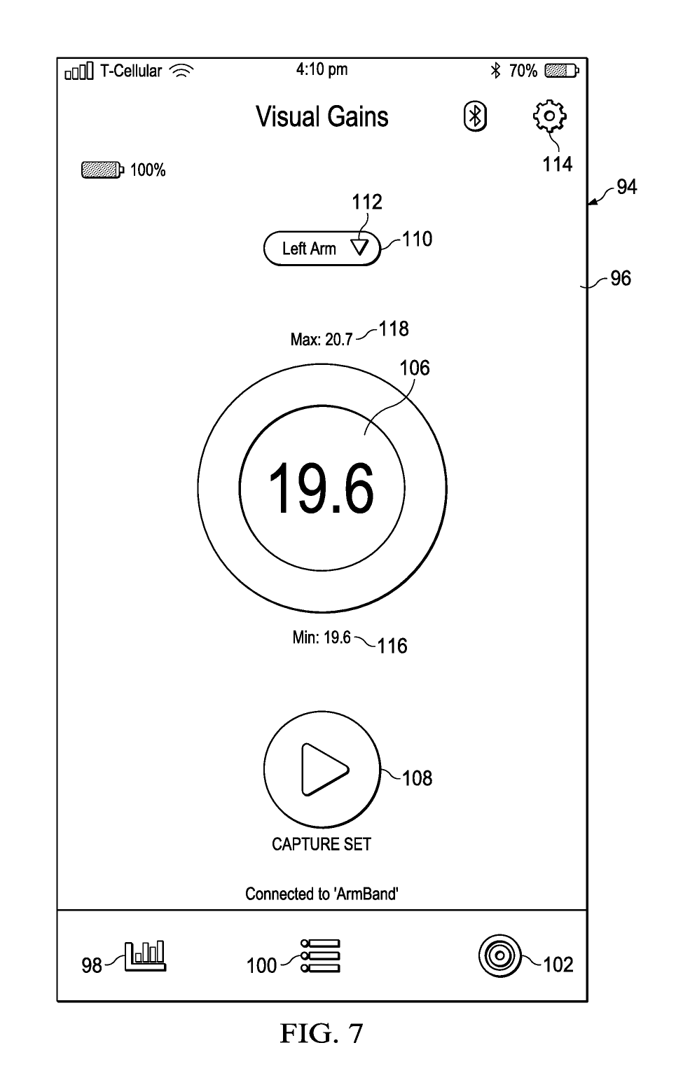

Referring to FIGS. 7-14, an enlarged view of the display 94 of mobile device 26 is shown with different examples of display screens provided by the native application on the mobile device 26. On the display 94, users may initially be provided with a login screen (not shown) where credentials are entered to access and enter information and create user profiles on the fitness band system 10 that is then accessible from any device by providing the same login credentials. It should be noted that while the following discussion describes the use of the interface and screen displays provided on the mobile device 26, the same or similar display screens and interface configurations, with all or at least a portion of the same information, may be provided on the display 84 of the electronic device 18 of the fitness band 12. Input devices, such as the control buttons 86, 88, 90, 92, may be used to similarly provide instructions or commands corresponding to those discussed for the mobile device 26, as well.

Furthermore, instructions or commands input into either the mobile device 26 or the fitness band 12, such as an instruction to begin measuring, may be simultaneously transmitted to the other device. Thus, if the user inputs an instruction to the mobile device 26 to begin measuring, the instruction will be transmitted to the fitness band 12, which will begin measuring. In this way, the mobile device 26 can be used to control the operation of the fitness band 12. Likewise, instructions or input into the fitness band 12, such as to display goals, may be similarly transmitted to the mobile phone 26 so that such goals are presented on both the fitness band 12 and mobile device 26.

In recording a body part measurement, the fitness band 12 is secured around a particular body part to measured, such as the bicep of the user 16, shown in FIG. 1. An appropriate band spacer 74A, 74B, or 74C may be selected and used to provide the necessary length for the strap assembly 20 so that it can be secured around the body part. In securing the fitness band around the body part, the circuit insert 72 of the band spacer 74 is inserted into one of the openings or apertures 70 to make contact with the band position sensor circuit 68. The choice of band spacer 74 and position of the circuit insert 72 into one of the apertures 74 of the strap assembly 20 will be selected to provide a fairly snug fit so that there is little slack that would interfere with accurate measurements. In some cases, the stretch sensor 60 may be stretched or expanded slightly, with the elastic material of the sensor 60 providing a contracting force so that the strap assembly 20 fits snugly around the body part being measured. Because the length of the band spacer 74 and other components of the strap assembly 20 are known, the spacing or distance of each of the apertures 70 of the position sensor 68 is known, the position of the insert 72 on the band spacer 74 is known, and the amount of deformation of the stretch sensor 60 is known, when the strap assembly 20 is secured to and worn on the body part of the user, the length of the entire strap assembly 20 can be calculated and determined. This will correlate to the circumferential dimension of the body part on which the strap assembly 20 is secured.

Length or dimension information for the strap assembly 20 may be immediately provided to the electronic device 18 and displayed in real time on the display, such as at 104 on the display 84 (FIG. 2) and/or at 106 on the display 94 of the mobile device 26 (FIG. 7). Because the components of the strap assembly 20 other than the stretch sensor 60 are non-stretchable or undergo very little lengthwise stretching under normal use, only the stretch sensor 60 changes dimension. Data regarding the degree of stretch or dimension of the stretch sensor 60 is provided to the processor 24 and, coupled with the known lengths and/or positions of the components of the strap assembly 20, an output reflecting the current dimension of the body part on which the fitness band 12 is worn can be provided. The current dimension can be immediately displayed in real time on the displays 84, 96 at 104, 106, respectively, so that the user can see real-time information or data relating to the dimensions of the body part being measured. Thus, changes in dimension of the stretch sensor 60 can be used to measure changes in the dimensions of the body part on which the device 12 is worn.

FIG. 7 shows an exemplary screen display 96 that is ready to begin recording measurements for a particular body part. As discussed, a similar screen may be provided on the display 84 of the fitness band 12, as well. In the embodiment shown, a set of different selection icons or buttons 98, 100, 102 are provided at the bottom of the screen 96. As an example, the control buttons 86, 88, 90, 92, may also correspond to any one of the selection icons 98, 100, 102. The screen 96 also includes a capture input icon or button 108. This may also correspond to one of the control buttons 86, 88, 90, 92. A body part indicator and menu 110 with a dropdown menu arrow 112 may be used to input the particular body being or to be measured. In the example shown, the left arm indicator is being displayed at 110. This indicates that the left arm has been selected to be the body part of interest. By using the dropdown arrow 112, a selection of different body parts may be provided and selected from the menu. Once selected, the body part (e.g., left arm) will be indicated in the indicator 110. The user may input the various body part selections into the menu or the menus of the devices may be pre-populated with the selections by the native application. On screen 96, the capture button 108 may be provided with an appearance to indicate capturing has started or not started.

An indication may also be provided on the screen 96 to show that the mobile device 26 is connected or paired with the fitness band 12. A settings selection icon 114 may allow the user to launch and access a pairing screen (not shown) for entering instructions or commands for pairing the mobile device 26 with the fitness band 12.

Also provided on the screen 94 are minimum and maximum measured dimensions 116, 118 during a selected interval or session.

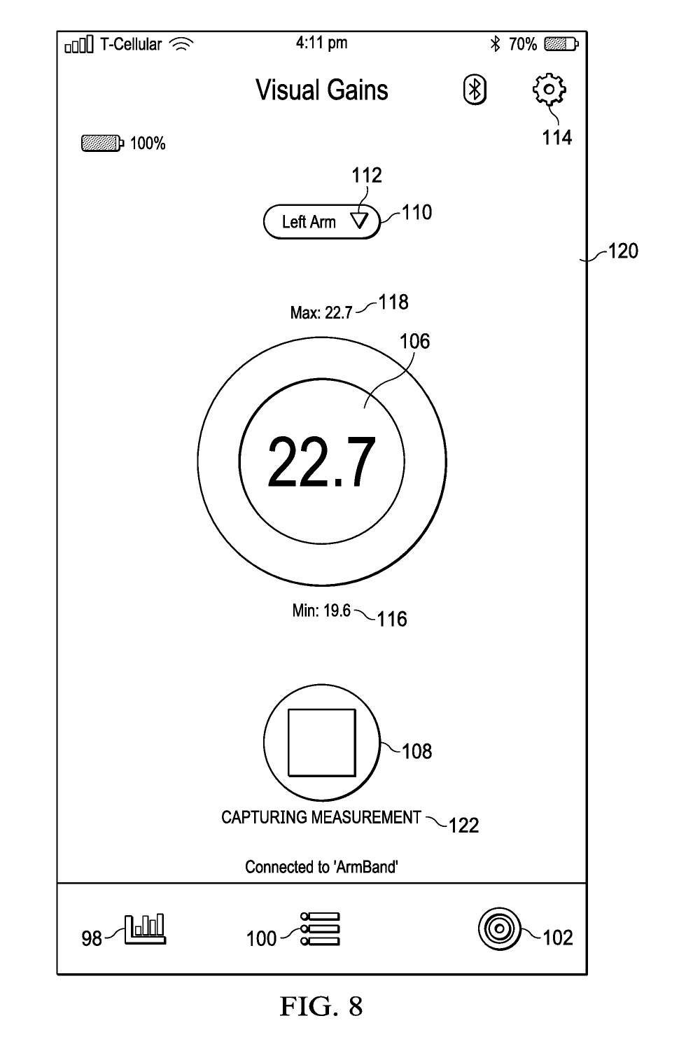

When the capture button 108 is activated, the fitness band will begin measuring and recording a measurement session. This is shown on the screen 120 of FIG. 8 with an indication being displayed at 122. The screen 120 is similar to the screen 96, with similar components or elements being labeled with the same reference numeral. And unless otherwise described or is apparent from its context, similar components or elements may be labeled with the same reference numeral for each of the different display screens of FIGS. 7-14 of the mobile device display 94. The appearance of the capture button 108 also may be changed to show that session measurements are being captured.

To end capturing of the measuring session, the capture button 108 may be actuated again. The measurement data may be stored in the memory 32 and/or persistent storage 32 of the data processing system 24 of the fitness band 12 and/or mobile device 24. It may also be transmitted and stored at a remote location, such as a remote server or computer (e.g., cloud storage), where the data may be accessed using the fitness band 12 and/or mobile device 26. As shown in FIG. 8, a minimum and maximum measurement 116, 118 are displayed for the session.

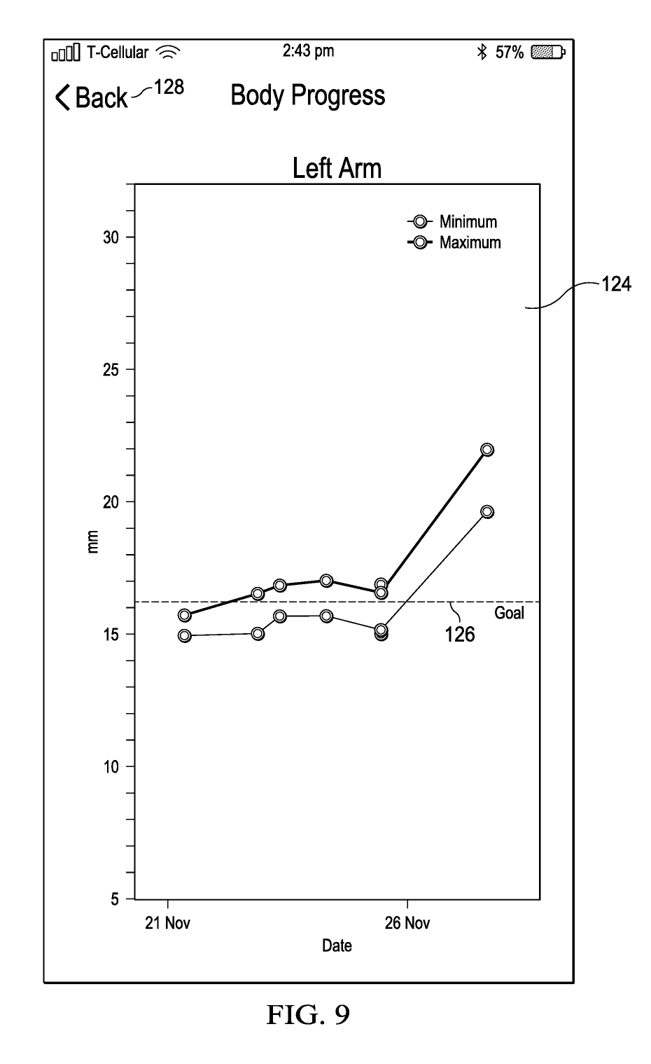

If the user wishes to review a charted history of minimum and maximum measurements over various sessions, the user may select the charted history access icon 98. This launches a charted history screen 124 (FIG. 9). The screen 124 shows a graphical representation of minimum and maximum measured body part dimensions during past sessions. A goal indicator 126 indicative of a user's measurement goal, which the user may input, is also displayed. A navigation button 128 allows the user to close or return to the previous screen or to another screen of the display 94. Again, similar displays and navigation can be provided on the electronic device 18 of the fitness band 12, as well.

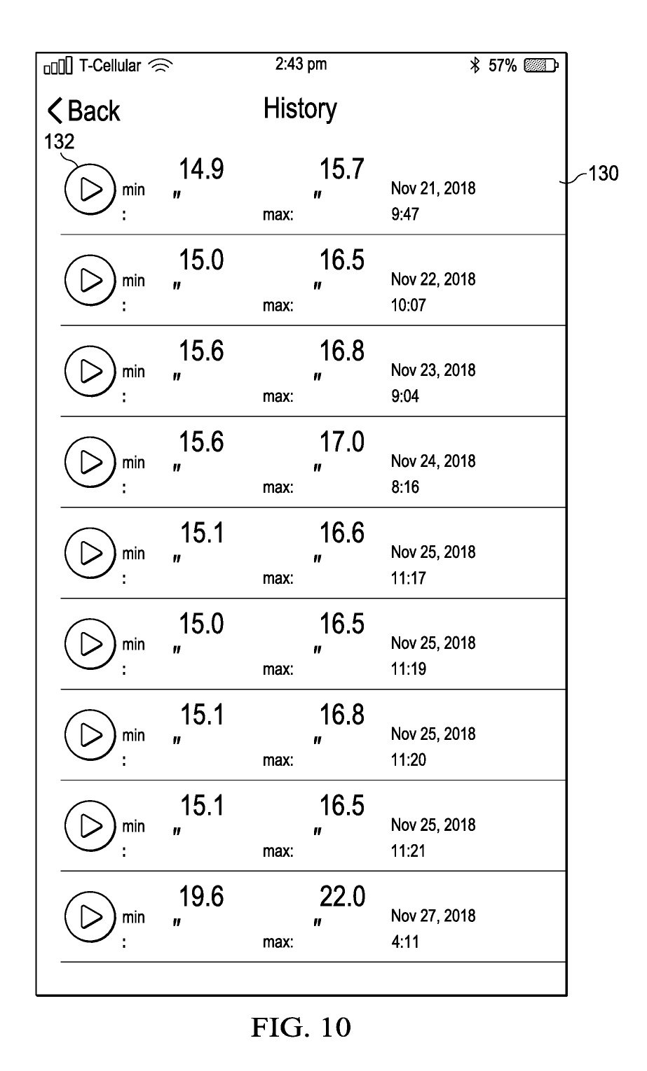

The user may review more detailed information for the captured sessions by actuating the history access icon 100 for the body part selected at 110 (FIG. 7). This launches a history screen 130 of FIG. 10. The history screen 130 shows individual data for each of the sessions captured with the system 10. Data for each session may include the minimum and maximum measurements, the date and time the session began or ended, duration of the session, etc. A replay button icon 132 may be provided with each listed session.

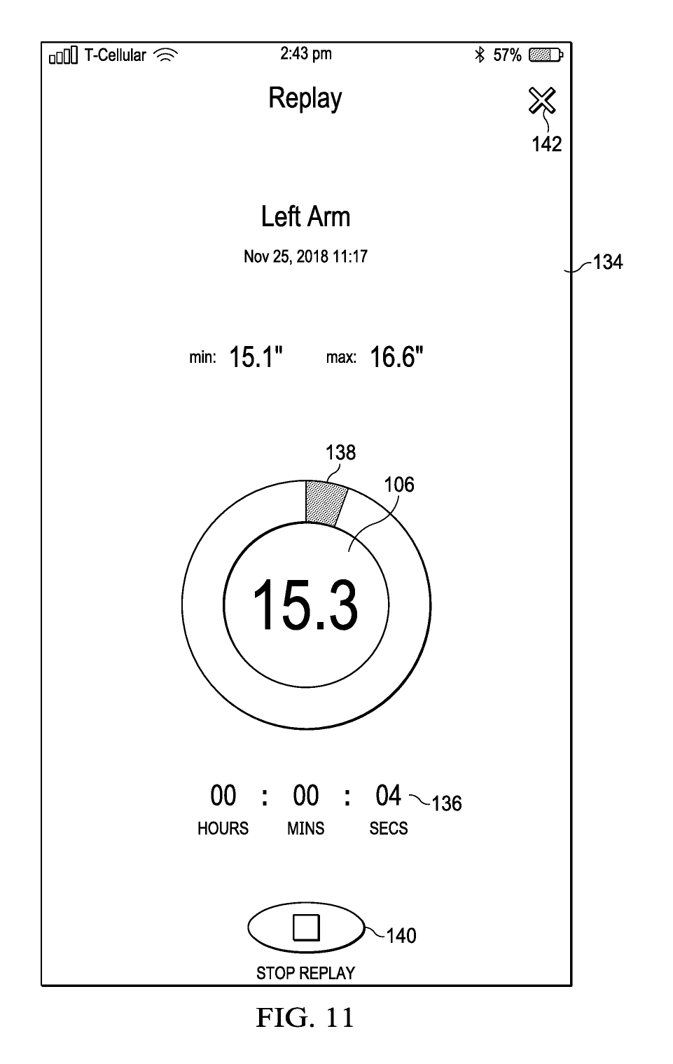

Actuating the replay icon 132 for a particular session launches a session replay screen 134, as shown in FIG. 11. The replay screen 134 shows various information from the measurement session. This includes the minimum and maximum measurements of the session, the body part being measured, and the date and time of the session. A time correlated measurement 106 is displayed that correlates to the elapsed time 136 during recording of the measurement session. As shown in FIG. 11, during the replay of the session, a measurement of 15.3 inches was measured at 4 seconds into the session. The user can watch a video of the changes in measurements at 106 during replay. A progress bar 138 can be provided to indicate the amount of time that has elapsed during replay between the start and end of the measurement session. The progress bar 138 can be a various configurations, such as straight, sloped, circular, etc. In the embodiment shown, the progress bar 138 may track a circle or loop with the progress bar moving 360 degrees clockwise or counter clockwise around the circle between the start and finish of the recorded session. In other embodiments, it may move inward or outward from the center our perimeter of the circle. Other configurations for showing progress of the recorded playback may also be used.

In particular applications, the progress bar 138, or other control mechanisms, may be used to advance or reverse the replay of the recorded session. Thus, for example, a user may manually move or manipulate the progress bar 138 forward or back to navigate to the different measurements over the duration of the session.

A stop/start replay icon button 140 is provided on the screen 134 to start and stop the replay. In some embodiments, manipulation of the progress bar 138 or other areas or controls of the screen 134 may start and stop replay. For instance, if during replay the user touches the progress bar 138, the replay may be stopped. Similarly, manipulating the progress bar 138 again may restart the replay.

A further navigation button 142 may be provided on the screen 134 to close or return to the previous screen or another screen.

If the user wishes to review the session history or chart history of another body part, the user may navigate to a screen having the body part indicator icon 110. By using the dropdown menu arrow 112, a list of different body parts is displayed and the desired body part can be selected. The selected body part will then be displayed at 110. Retrieving session chart history and session history for such newly selected body part is the same as that previously described.

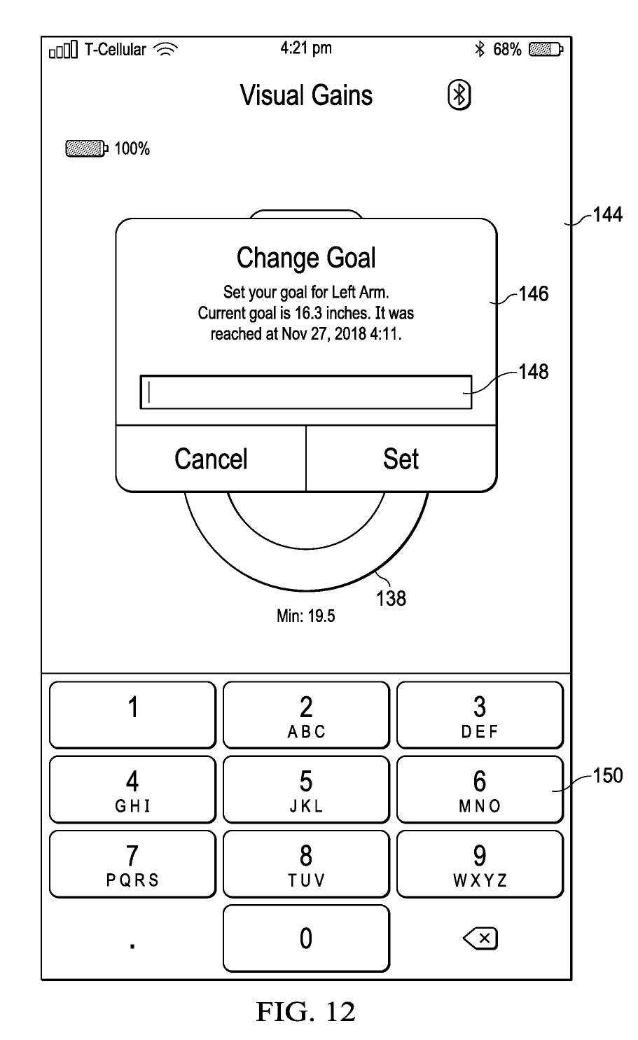

Setting goals for a particular body part may be achieved by actuating the goal setting icon button 102 (FIG. 7). This launches a goal setting screen 144, as shown in FIG. 12. At the goal setting screen 144, a “change goal” interface 146 appears. In the embodiment shown, the interface 146 may list the current set goal and when and/or if it may have been last met. Other information may also be provided, such as an indication of the body part for which the goal applies. A goal entry field 148 allows the user to set a desired goal using a launched keyboard 150. The user may then set the new goal or cancel the entered goal, using set or cancel icon buttons 152, 154, wherein the goal may revert back to the previously set goal data. Upon setting or canceling the “change goal” interface, the previous screen or another screen of the display 94 may appear. Setting goals for different body parts is achieved by selecting the body part indication and selection icon 110 and by using the dropdown menu arrow 112 to navigate to and select the particular body part for which the goal is to be set.

Once the goal is set, it may appear on various screens of the display 94, such as the goal line 126, shown on the chart history screen 124 of FIG. 9, and/or on the display 84 of the fitness band 12, such as at 156 (FIG. 2). Additionally, when a set goal has been achieved, a notification may be provided by the fitness band 12 and/or mobile device 26 to notify the user that the goal has just been reached during the measurement activity. This may be a visual, haptic, and/or audible notification. Such a feature, as well as other features, may be selectively enabled or disabled by the user. For haptic notifications, a haptic feedback device may be included with the fitness band device 12, which may provide a vibration or other haptics.

Settings for the native application 52 of the mobile device 26, fitness band 12, and/or system 10, may be accessed by actuating settings icon button 114. Upon actuating, a settings menu screen 158 is launched. The settings menu screen 158 has various setting selections. These may include, but are not limited to, band spacer setup selection 160, band measurement adjustment 162, device connectivity selection 164, such as for pairing the mobile device 26 with the fitness band 12. Other selections and options may also be provided on the settings menu page 158.

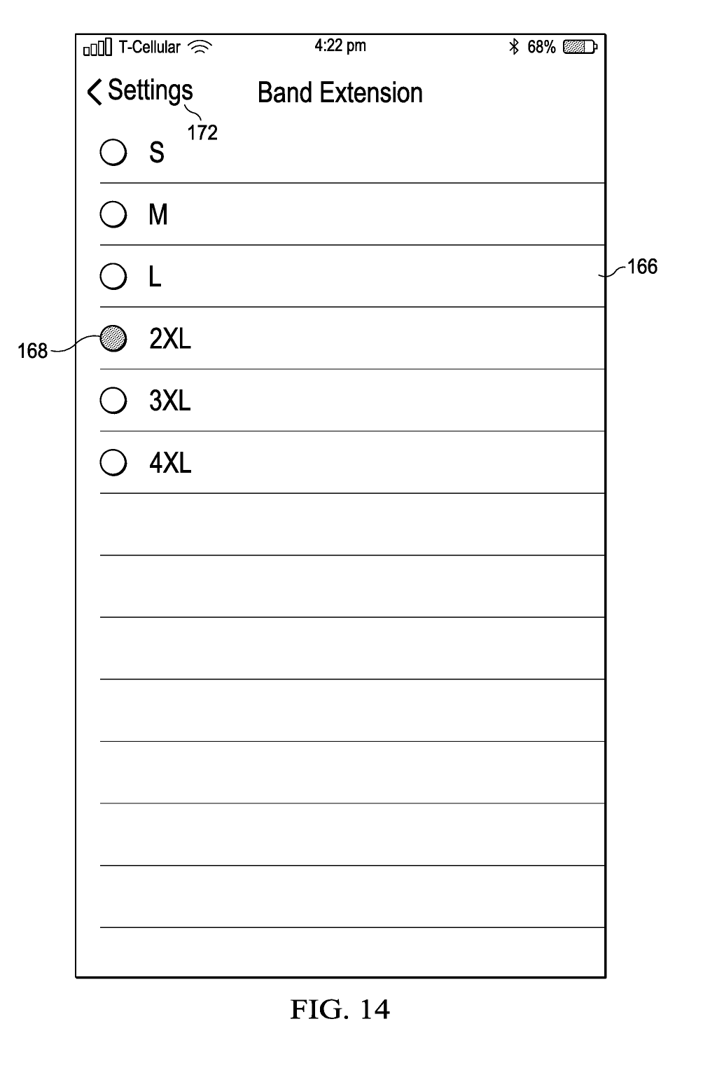

By selecting the band spacer setup selection 160, a band spacer selection page 166 is launched on the display 94, as shown in FIG. 14. Here, different selections for the different size band spacers 74A, 74B, 74C, etc. are provided. Here a 2XL band spacer is shown as being selected at 168. As shown in FIG. 2, identifying information 170 related to the selected band spacer may be displayed on the display 64 of the fitness band 12. This informs the user that the correct band spacer information has been entered. By selecting one of the different size band spacers 74, data is provided to the data processor 24 of the particular band spacer 74 being used. In some embodiments, an autodetect feature may be provided so that the system 10 or band device 12 can automatically detect the particular band spacer being used. Knowing the particular band spacer being used allows the data processor 24, combined with position data for the circuit insert 72 and data from the stretch sensor 60, to determine and provide a measurement output relating to the dimension of the strap assembly 20, which correlates to measurement of the body part being measured. A navigation icon 172 may be provided on the screen 166 for navigating to the previous or a different screen.

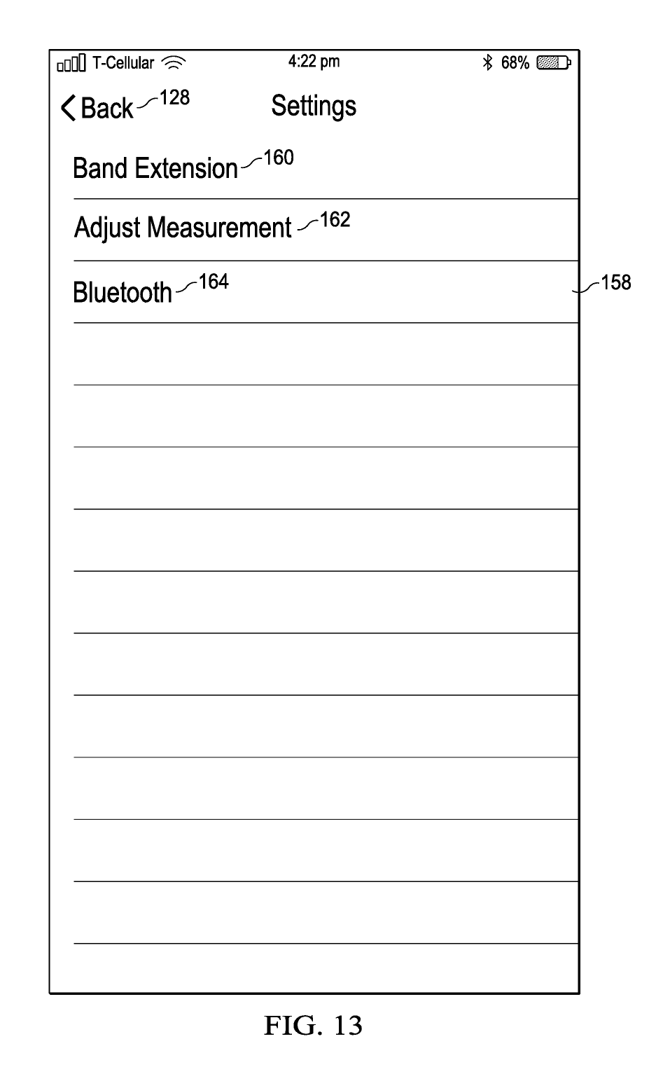

If there are errors in the measurement or the fitness band device is providing an inaccurate measurement, the user may adjust the measurement by selecting the band measurement adjustment selection 162 on screen 148 (FIG. 13). Upon selection, features for adjusting the measurement readout or recalibrating the device may be made.

The device connectivity selection 164, provides an interface on the display 94 wherein the user can locate and pair the mobile device 26 with a particular fitness band 12 or other devices.

As can be appreciated, the fitness band 12, with or without any mobile device 26, can be used to perform measurements of a body part of a user. The measurement will typically be the circumference of a person's limb, such as the forearm, bicep, calf, thigh, etc. Other body parts may also be measured. While the fitness band has particular application to measurement of human body parts, it may also be used for measuring body parts of animals or other objects, as well, for fitness or other non-fitness purposes.

In certain applications, more than one fitness band may be worn by a user at a time. Thus, the fitness band may be worn on two different body parts, such as both biceps, or at different positions on the same body part. In such cases, mobile device 26 may be simultaneously paired with or connected to more than one fitness band and the screens and interfaces the mobile device may account for and accommodate displaying information for the two or more fitness bands being used.

The fitness band 12 will typically be used during particular activities that may have an effect on the dimensions of the particular body part being measured. Oftentimes this will be weightlifting or strength training activities, although it may be used during other activities as well. In one example, the fitness band 12 may be positioned around one or both biceps of a user and used while the user is performing a set of dumbbell or barbell curls. The fitness band allows the user to see the size and changes in size of the body part in real time, as well as during review and playback.

As discussed previously, the orientation of the display may be also varied. If the user is using the fitness band in front of a mirror, the user may reverse the image of the display 84 so that it becomes readable while looking at the display image in the mirror. If the user does not want anyone else to view the display 84 of the fitness band 12 during use, the user may selectively turn the display 84 to an incognito mode where no information or only selected information is displayed on the display 84.

In certain embodiments, the fitness band system 10 may be used in connection with various connected communities or social media platforms. This may include the sharing of measurement results with a community of users. Games and competitions within a community of users may also be performed, where measurement data is used as part of such activities.

Other functionality may be included with the fitness band 12, as well. This may include typical fitness band or tracker functionality, such as GPS tracking, step counting, heart rate monitoring, sleep statistics, time and date display, haptic feedback, music and audio play, etc. The fitness band 12 may even be incorporated with or configured as a smart watch, with all the functionality of a smart watch (e.g., text messaging, emailing, web surfing, phone calling, accessing an electronic personal assistant, fitness tracking, photo display, video display, GPS tracking, step counting, heart rate monitoring, sleep statistics, time and date display, haptic feedback, music and audio play, etc.) coupled with the capability of performing body measurements, as has been described.

While the invention has been shown in only some of its forms, it should be apparent to those skilled in the art that it is not so limited, but is susceptible to various changes and modifications without departing from the scope of the invention. Accordingly, it is appropriate that the appended claims be construed broadly and in a manner consistent with the scope of the invention.

I claim:

1. A wearable fitness band system that measures a body part change in dimension, the device comprising:
a wearable fitness band device comprising strap assembly that is configured for being wrapped around the circumference of a body part to be measured, the strap assembly having a sensor that senses a change in dimension of the strap assembly while the strap assembly is wrapped around the body part and provides a sensor output;
a processing unit coupled to the sensor for receiving the sensor output and providing a measurement output; and
a display that receives the measurement output and provides a measurement output reading correlating to the measurement output.

2. The system of claim 1, wherein:
the sensor is an elastomeric stretch sensor.

3. The system of claim 2, wherein:
the elastomeric stretch sensor constitutes a section of the strap assembly, with the remainder of the strap assembly being non-stretchable.

4. The system of claim 1, wherein:
the display is provided on at least one of a display of the wearable fitness band device and a separate mobile device.

5. The system of claim 1, wherein:
the wearable fitness band device further comprises a band position sensor that receives a position coupling when the strap assembly is wrapped around the body part to facilitate determining a reference dimension of the strap assembly.

6. The system of claim 1, wherein:
wearable fitness band device further comprises a removable band spacer that facilitates selective shortening and lengthening of the strap assembly.

7. The system of claim 5, further comprising:
a removable band spacer that facilitates selective shortening and lengthening of the wearable band assembly, and wherein the position coupling is provided on the band spacer.

8. The system of claim 1, wherein:
the system provides a real-time measurement output that is displayed on the display.

9. The system of claim 1, wherein:
the display selectively provides at least one of a reverse, a rotated, and an upside-down image display.

10. The system of claim 1, wherein:
the wearable fitness band device accommodates body parts ranging from 8 inches to 50 inches in circumference.

11. The system of claim 1, wherein:
the wearable fitness band device measures a change in dimension of from 0.01 inch or more.

12. The system of claim 1, wherein:
the wearable fitness band device provides text at least one of messaging, emailing, phone calling, web surfing, accessing an electronic personal assistant, fitness tracking, photo display, video display, GPS tracking, step counting, heart rate monitoring, sleep statistics, time and date display, haptic feedback, music play, and audio play.

13. A wearable fitness band system that measures a body part change in dimension, the system comprising:
a wearable fitness band device comprising a strap assembly that is configured for being wrapped around the circumference of a body part to be measured, the strap assembly comprising:
a length of elastomeric stretch sensor that senses a change in dimension of the strap assembly when the elastomeric stretch sensor is expanded and retracted;
at least one removable band spacer having a known length, the band spacer having a position coupling fixed thereon; and
a band spacer position sensor that is configured to engage the position coupling of the at least one removable band spacer at different positions along the length of the band spacer position sensor; and
a processing unit coupled to the strap assembly for providing a measurement output in response to receiving an output signal from the elastomeric stretch sensor; and
a display coupled to the processing unit that receives the measurement output and provides an output on the display correlating to the measurement output.

14. The system of claim 13, wherein:
the display is provided on at least one of a display of the strap assembly and a separate mobile device.

15. The system of claim 13, wherein:
there are at least two interchangeable band spacers of different lengths that facilitate shortening and lengthening of the strap assembly.

16. The system of claim 13, wherein:
the system provides a real-time measurement output that is displayed on the display.

17. The system of claim 13, wherein:
the display selectively provides at least one of a reverse, rotated, and upside-down image display.

18. The system of claim 13, wherein:
the strap assembly accommodates body parts ranging from 8 inches to 50 inches in circumference.

19. The system of claim 13, wherein:
the system measures a change in dimension of from 0.01 inch or more.

20. A method of measuring a change in circumference of a body part comprising:
positioning a wearable fitness band device that is configured for being wrapped around the circumference of a body part to be measured;
sensing with a sensor of the wearable band device a change in dimension of the wearable fitness band device while the wearable band is wrapped around the body part;
providing a sensed output that correlates to the sensed change in dimension; and
displaying on a display device a measurement output based upon the sensed output.
